(19) United States
(12) Patent Application Publication
(10) Pub. No.: US 2004/0153091 A1
(43) Pub. Date: Aug. 5, 2004

(54) Sizing plate and sizing plate extraction

(76) Inventors: Marvin Figueroa, Warsaw, IN (US); Scott Dykema, Warsaw, IN (US)

Correspondence Address: BAKER & DANIELS, 111 E. WAYNE STREET, SUITE 800, FORT WAYNE, IN, 46802

(21) Appl. No.: 10/357,991

(22) Filed: Feb. 4, 2003

(51) Int. Cl. A61B 17/58

(52) U.S. Cl. 606/102

(57) ABSTRACT

An orthopaedic instrumentation assembly for determining the appropriate prosthetic proximal tibial component for a particular patient. The instrumentation assembly includes an apparatus for extracting the sizing plate from the proximal tibial end to allow for implantation of the prosthetic proximal tibial component. The sizing plate includes a first surface adapted for connection to the bone end and an opening through the plate having an inner periphery and an outer periphery defining at least one shoulder. The plate extractor includes an extraction key shaped to pass through the sizing plate opening and slidingly engage the shoulders. Depending from the extraction key is an instrument adaptor such as a threaded member for attaching a T-handle or slap-hammer instrument. A second surface of the sizing plate opposite the first surface includes a plurality of counterbored holes for receiving fixation pins. The sizing plate can be fastened to the proximal tibial end without pin cap heads, pegs, or other devices protruding from the second surface. After the trial is complete, the plate extractor can be engaged with the shoulders of the sizing plate, and force applied on the plate extractor in a direction away from the proximal tibial end to extract the sizing plate and fixation pins from the tibial end without damaging the tibial plateau.

### BACKGROUND OF THE INVENTION

1. Field of the Invention

The present invention relates to orthopaedic instrumentation, and, more particularly, to a sizing plate for determining the appropriate prosthetic proximal tibial component for a particular patient as well as an apparatus for extracting the sizing plate to allow for implantation of the prosthetic proximal tibial component.

2. Description of the Related Art

Generally, the knee is formed by a pair of condyles at the distal portion of the femur, the lower surfaces of which bear upon the correspondingly shaped proximal surface plateau of the tibia, with a pair of menisci positioned therebetween, with each condyle having a meniscus associated therewith. The femur and the tibia are connected by means of ligaments such as, the posterior cruciate ligament, the lateral collateral ligament, the medial collateral ligament, and the anterior cruciate ligament. These ligaments provide stability to the knee. Similarly, a prosthetic knee joint includes a femoral component, and a tibial component, with a prosthetic bearing, or a meniscal component positioned therebetween.

In a broad sense, prosthetic knee joints can be considered either constrained or unconstrained. For the purposes of this discussion, constrained prosthetic knees include femoral and tibial prosthetic components which are mechanically linked or constrained to each other by a hinge structure. An unconstrained prosthetic knee includes femoral and tibial components which are not mechanically linked. An unconstrained knee utilizes the patient's existing ligaments to provide joint stability. With this in mind, constrained prosthetic knees have particular applicability to cases in which a patient has experienced ligament loss and/or the existing ligaments do not provide adequate support and stability to the knee.

Tibial components of a prosthetic knee can be formed as a one-piece configuration in which the prosthetic meniscal component is an integral component of the tibial tray or tibial plateau of the prosthetic knee. Various other prosthetic knees utilize modular meniscal components separate from the tibial component. Devices utilizing modular meniscal components include those in which the meniscal component is fixed to the tibial tray portion of the prosthetic tibial component and is incapable of movement relative thereto. Alternative devices utilize a modular meniscal component capable of movement relative to the tibial tray. Devices in which relative rotational movement occurs between the meniscal component and the tibial component are typically referred to as rotating bearing knees. Rotating bearing knees thus allow movement between the bearing, i.e., meniscal component and the tibial tray as well as movement between the femoral component and the tibial bearing.

When revising or newly implanting a tibial prosthesis, a platform on the proximal tibia is first prepared by removing the failed tibial components, and/or resecting the proximal tibia. In some cases, the intramedullary canal is reamed to further prepare the tibia to receive a prosthetic tibial component. After the tibia is prepared, a provisional tibial sizing plate is used to determine the proper size prosthetic tibial tray and meniscal component. In use, the sizing plate is placed on the proximal tibia and checked for size.

After the proper sizing plate is selected, the sizing plate is held in position by fixation pins placed through the tibial plate and into the proximal tibia. The fixation pins generally have cap heads that remain extended above the surface of the sizing plate so that the pins can be extracted upon completion of the trial. Alternatively, the sizing plate may have a peg or other component extending from the proximal surface of the sizing plate so that the plate may be later pulled from the tibial plateau.

With the proper sizing plate selected and held in place, a drill or other guide can be located in receiving guides defined on the upper surface of the sizing plate and the proximal tibia can be drilled and/or broached to accommodate tibial stems or other supporting components of the prosthetic tibial component.

After the proximal tibia is fully prepared, and with the sizing plate in position atop the proximal tibia, remaining trial prosthesis components can be assembled to perform a trial reduction to test range of motion and for soft tissue and ligament checks.

After the trial reduction is complete, the various prosthetic trial components must be removed, including the tibial sizing plate. The sizing plate should be removed without damaging the proximal tibia. For tibial sizing plates having securing pins with cap heads or pegs extending above the plate surface, the plate can be pulled from the proximal tibia without damaging the bone stock. However, for some tibial components, the proximal surface of the sizing plate must be without protrusions so that the prosthetic meniscal component can be properly received atop the sizing plate. This is true for certain fixed bearing components and is particularly true of rotating bearing components in which the meniscal component moves relative to the tibial sizing plate during a trial reduction.

One option for eliminating protrusions from the proximal surface of the sizing plate is to countersink the pin cap heads into the tibial sizing plate, thus providing a flat proximal sizing plate surface for reception of the prosthetic meniscal component. However, countersunk pin cap heads require the use of a prying instrument positioned between the distal surface of the sizing plate and the proximal tibia to remove the sizing plate and the retaining pins from the tibial plateau. This procedure can easily lead to damage of the proximal tibia.

What is needed in the art is a tibial sizing plate having a proximal surface adapted to accommodate a rotating bearing, and a cooperating apparatus for removing the tibial plate and retaining pins without application of tools and harmful pressure to the proximal tibia.

### SUMMARY OF THE INVENTION

The present invention provides an orthopaedic instrumentation assembly including a sizing plate for determining the appropriate prosthetic proximal tibial components for a particular patient as well as a plate extractor for extracting the sizing plate from the proximal tibia upon completion of a successful trial reduction.

The sizing plate includes a distal surface adapted for connection to the tibial plateau and a proximal surface for receiving a prosthetic meniscal component. The sizing plate includes holes counterbored from the proximal surface for receiving cap head fixation pins to hold the sizing plate to the proximal tibia during a trial reduction. Because the fixation pin holes are counterbored, the cap heads of the pins can be flush or below the proximal surface of the sizing plate so that the sizing plate can receive both fixed or rotating bearing meniscal components requiring a flat tibial plateau. The sizing plate further includes a central opening having a shoulder.

The plate extractor includes an extraction key and an instrument adaptor depending from the extraction key. The extraction key is shaped so that it can be inserted from the plate's proximal surface through the central opening of the sizing plate, and then moved so that at least a portion of the extraction key abuts the shoulder of the central opening, whereby the shoulder prevents the extraction key from being moved relative to the sizing plate in a direction generally parallel to the longitudinal axis of the tibia, i.e., a direction generally perpendicular to the proximal surface of the sizing plate. The sizing plate and the countersunk fixation pins can then be removed from the proximal tibia by pulling the plate extractor away from the tibial plateau in a direction generally parallel to the longitudinal axis of the tibia, i.e, a direction generally perpendicular to the proximal service of the sizing plate. When force is applied in this manner, the plate extractor acts against the shoulder of the central opening of the sizing plate to transfer force from the extraction key to the sizing plate and the fixation pins. With the construction of the present invention, the sizing plate and the fixation pins may be removed without compromising the tibial bone stock.

The invention, in one form thereof, comprises an orthopaedic instrumentation assembly for at least partially preparing an end of a bone for receiving a prosthetic component, the instrumentation assembly including a plate extractor having an extraction key and a pin depending from a surface of the extraction key, with the pin defining an instrument adaptor. The instrumentation assembly further includes a sizing plate having a first surface adapted for placement atop the bone end, and a second surface opposite the first surface. The sizing plate further includes an opening having an inner and outer periphery defining at least one shoulder. The opening in the sizing plate is sized to accommodate passage of the extraction key therethrough.

The invention, in another form thereof, comprises an orthopaedic instrumentation assembly for at least partially preparing an end of a bone to receive a prosthetic implant. The instrumentation assembly of this form of the present invention includes a sizing plate having a first surface adapted for placement atop the bone end, and a second surface opposite the first surface as well as extraction means for removing the sizing plate from a secured position atop the bone end, without directly contacting the bone end.

The invention, in another form thereof, comprises a method for removing an orthopaedic plate member from a bone end. The method of this form of the current invention includes the steps of: inserting a plate extractor key into an opening defined through the plate member, moving the extractor key into position to engage at least a portion of the extractor key in a recess defined between the plate member and the bone end, and pulling the extractor key away from the bone end.

Advantageously, the present orthopaedic instrumentation assembly provides an apparatus and method for fixing a trial sizing plate to a tibial plateau such that the proximal surface of the sizing plate is free from protrusions in the area that receives the prosthetic meniscal component. Additionally, the assembly provides an apparatus and method for removing the sizing plate and fixation pins from the proximal tibia without applying pressure to or damaging the tibial plateau.

### BRIEF DESCRIPTION OF THE DRAWINGS

The above-mentioned and other features and advantages of this invention, and the manner of attaining them, will become more apparent and the invention itself will be better understood by reference to the following description of embodiments of the invention taken in conjunction with the accompanying drawings, wherein:

FIG. 1 is an exploded perspective view of a sizing plate and cooperating plate extractor of the present invention;

FIG. 2 is a proximal elevational view of a tibial sizing plate of the present invention;

FIG. 3 is a proximal elevational view of a first embodiment plate extractor of the present invention;

FIG. 4 is a posterior elevational view of the plate extractor of FIG. 3;

FIG. 5 is a proximal elevational view of the sizing plate of FIG. 2 with a plate extractor of the present invention positioned for removal of the sizing plate, with the plate extractor shown in sectional view taken along line 5-5 in FIG. 4;

FIG. 6 is a partial sectional view taken along lines 6-6 of FIG. 5;

FIG. 7 is a proximal elevational view of a second embodiment plate extractor of the present invention; and

FIG. 8 is a posterior elevational view of the plate extractor shown in FIG. 7.

Corresponding reference characters indicate corresponding parts throughout the several views. The exemplifications set out herein illustrate preferred embodiments of the invention, and such exemplifications are not to be construed as limiting the scope of the invention in any manner.

### DETAILED DESCRIPTION OF THE INVENTION

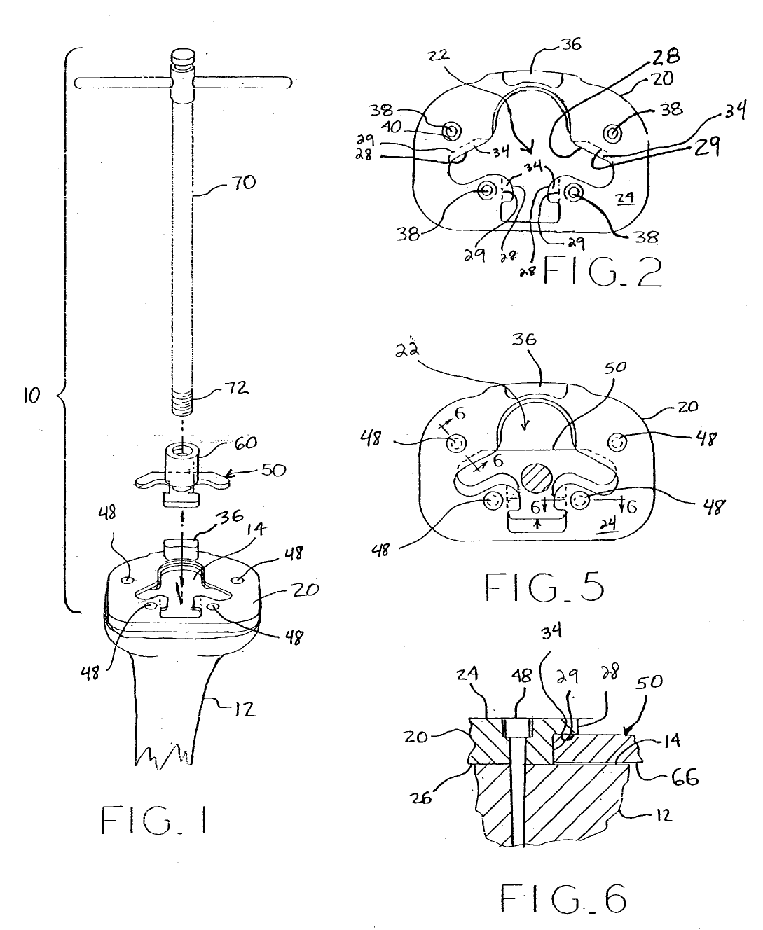

An exemplary embodiment sizing plate instrumentation assembly 10 is shown in FIG. 1. The assembly includes tibial sizing plate 20, plate extractor 50, and extractor tool 70. Tibial sizing plate 20 is utilized to determine the appropriate proximal tibial prosthetic component for a particular patient. Tibial sizing plate 20 includes distal surface 26, shown in FIG. 6, adapted for trial placement on proximal end 14 of tibia 12, shown in FIG. 1. Proximal tibia 14 is shaped with appropriate cutting instrumentation before tibial sizing plate 20 is attached thereto.

Referring to FIG. 2, tibial sizing plate 20 includes proximal surface 24 and opening 22 defined between proximal surface 24 and distal surface 26, shown in FIG. 6. Sizing plate 20 further includes rotation tab 36, shown in FIG. 1, depending from proximal surface 24. Rotation tab 36 is, in certain embodiments, utilized to guide rotation of a rotating meniscal component.

Holes 38 are defined through sizing plate 26 and include counterbores 40 (FIG. 2) adjacent proximal surface 24. Holes 38 receive fixation pins 48, as shown in FIG. 6, for fixing sizing plate 20 to tibia 12. Counterbores 40 allow the cap head of fixation pin 48 to be flush with (FIG. 6) or below proximal surface 24 of sizing plate 20, thus providing a flat proximal surface 24 free of protrusions.

Referring again to FIG. 2, inner periphery 28 and outer periphery 29 of opening 22 define shoulders 34 therebetween. Opening 22 is shaped to accept various bone shaping and resecting instruments for preparing tibia 12 to receive a final tibial implant. Opening 22 is also shaped to receive plate extractor 50 as shown in FIG. 5

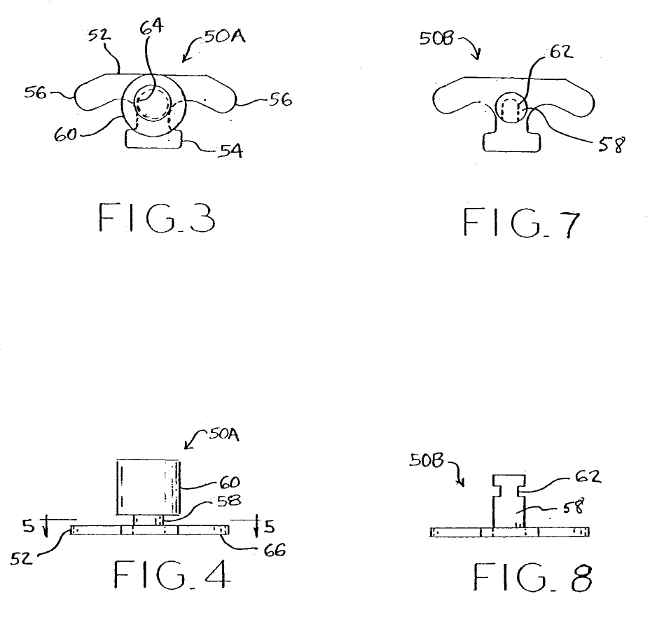

Referring to FIGS. 3 and 4, plate extractor 50A includes plate key 52, peg 58 depending from key 52, and instrument adaptor 60 depending from peg 58. Instrument adaptor 60 includes a threaded receptacle defined therein. FIGS. 7 and 8 show plate extractor 50B, having instrument adaptor neck portion 62 defined along peg 58.

Referring now to FIG. 3, extraction key 52 is a generally flat plate having wings 56 and foot 54 defining a shape capable of traversing opening 22 in sizing plate 20, and thereafter moving in a direction substantially parallel to proximal surface 24 of sizing plate 20 to position wings 56 and foot 54 of extraction key 52 under shoulders 34 (FIG. 2) of sizing plate 20 as illustrated in FIGS. 5 and 6. Referring to FIGS. 2 and 5, extraction key 52 passes through opening 22 of sizing plate. After distal key surface 66 (FIG. 4) of extractor key 52 is substantially flush with distal surface 26 of sizing plate 20, as shown in FIG. 6, plate extractor 50 can be slid anteriorly toward rotation tab 36 to position wings 56 and foot 54 under shoulders 34 of sizing plate 20 as illustrated in FIGS. 5 and 6.

After tibial plateau 14 is prepared with cutting or other instrumentation, the tibial plate size to be used is selected using tibial sizing plates 20 of various sizes. Earlier preparation of tibial plateau 14 may include leaving an intramedullary reamer or other instrument extending vertically from tibia 12 at tibial plateau 14. If such a reamer or other instrument is in place, opening 22 of sizing plate 20 is placed over the reamer and sizing plate 20 is then placed on and secured to tibial plateau 14. Tibial plate 20 can then be checked for size as compared to tibial plateau 14, and a different sizing plate 20 selected if required.

After sizing plate 20 has been properly located on tibial plateau 14, sizing plate 20 is preferably fixed in position using cap-headed fixation pins 48. As shown in FIG. 6, fixation pins 48 extend through holes 38 defined through sizing plate 20 and are pressed into proximate end 14 of tibia 12 until the cap head of fixation pin 48 is flush with or below proximal surface 24 of sizing plate 20. Any additional drilling and reaming required to prepare tibia 12 to receive the final tibial implant can be performed with bone shaping and resecting instruments positioned through opening 22 in sizing plate 20. After tibia 12 is fully prepared, trial components, including a trial prosthetic meniscal component, can be implanted and a trial reduction can be performed. Advantageously, proximal surface 24 of sizing plate 20 has no protrusions other than tab 36 depending upward from it, and thus provides a flat surface for both fixed or rotational bearing prosthesis components.

After trial prosthesis components and articulation have been evaluated and the components removed, sizing plate 20 can be removed from tibial plateau 14 in preparation for implantation of final implant components. To remove sizing plate 20 from tibial plateau 14, plate extractor 50 is attached at adaptor 60 to extraction tool 70, such as the T-handle shown in FIG. 1, or another instrument such as a slap-hammer. Extraction key 52 of plate extractor 50 is then inserted through opening 22 until distal surface 66, shown in FIG. 6, contacts tibial plateau 14 and/or is substantially flush with distal surface 26 of sizing plate 20. Then, as shown in FIG. 5, plate extractor 50 is slid anteriorly toward rotation tab 36, such that wings 56 and foot 54 of extraction key 52 engage shoulders 34 of sizing plate 20. As shown in FIG. 6, extraction key 52 is then in a position to apply a force to extraction shoulders 34, pulling sizing plate 20 away from tibial plateau 14, and thereby extracting fixation pins 48 from tibia 12. An upward force away from tibial plateau 14 along an axis parallel to the longitudinal axes of fixation pins 48 is applied to extraction key 52 by pulling extraction tool 70 away from tibial plate 14. Tibial sizing plate 20 and fixation pins 48 are thus removed from tibia 12 without directly applying an instrument to and potentially damaging tibial plateau 14.

Other shapes and engagement methods for selectively connecting plate extractor 50 to sizing plate 20 may alternatively be used. Preferably the force transmitted by plate extractor 50 to sizing plate 20 will evenly distribute an upward force through counterbores 40 to fixation pins 48. For example, as shown in FIGS. 2 and 5, shoulders 34, which transmit the upward force from extraction key 50 to sizing plate 20, are centrally located between holes 38 and are adjacent holes 38. Thus, an upward force applied to plate extractor 50 extracts fixation pins 48 evenly, keeping sizing plate 20 substantially parallel to tibial plateau 14 and extracting fixation pins 48 directly along the pins' longitudinal axes. By way of further example, a twisting motion on plate extractor 50, rather than a sliding motion, could be used to engage a differently arranged embodiment of sizing plate 20.

While this invention has been described as having exemplary embodiments and scenarios, the present invention can be further modified within the spirit and scope of this disclosure. This application is therefore intended to cover any variations, uses, or adaptations or the invention using its general principles. Further, this application is intended to cover such departures from the present disclosure as come within known or customary practice in the art to which this invention pertains and which fall within the limits of the appended claims.

What is claimed is:

1. An orthopaedic instrumentation assembly for at least partially preparing an end of a bone for receiving a prosthesis, said instrumentation assembly comprising:
a plate extractor having an extraction key and a pin depending from a surface of said extraction key, said pin defining an instrument adaptor; and
a sizing plate having a first surface adapted for placement atop the bone end, a second surface opposite said first surface and an opening therethrough having an inner and outer periphery defining at least one shoulder, said opening sized to accommodate passage of said extraction key therethrough.

2. The orthopaedic instrumentation assembly of claim 1 further comprising:
a plurality of fixation means for fixing said sizing plate to the bone end, and wherein said inner and outer periphery define a plurality of shoulders corresponding in number to said fixation means, said shoulders positioned adjacent said fixation means on said sizing plate.

3. The orthopaedic instrumentation assembly of claim 2, wherein said fixation means comprises:
a plurality of fixation pins, and wherein said sizing plate includes a plurality of apertures corresponding in number to said fixation pins.

4. The orthopaedic instrumentation assembly of claim 1, further comprising:
a plurality of fixation pins, said sizing plate having a plurality of apertures, said apertures having counterbores on said second surface of said sizing plate.

5. The orthopaedic instrumentation assembly of claim 1, wherein said instrument adaptor comprises a threaded instrument adaptor.

6. The orthopaedic instrumentation assembly of claim 1, wherein said instrument adaptor includes a neck portion formed on said pin, said pin having an outer periphery, said neck having an outer periphery, said outer periphery of said neck smaller than said outer periphery of said pin.

7. An orthopaedic instrumentation assembly for at least partially preparing an end of a bone for receiving a prosthesis, said instrumentation assembly comprising:
a sizing plate having a first surface adapted for placement atop the bone end, and a second surface opposite said first surface; and
extraction means for removing said sizing plate from a secured position atop the bone end, without directly contacting the bone end.

8. The orthopaedic instrumentation assembly of claim 7, wherein said sizing plate has a central opening, said central opening having an inner and an outer periphery defining a plurality of shoulders, and wherein said extraction means comprises an extraction key shaped to pass through said opening and selectively engage said plurality of shoulders.

9. The orthopaedic instrumentation assembly of claim 8, further comprising an instrument adaptor depending from said extraction key.

10. The orthopaedic instrumentation assembly of claim 7, further comprising:
a plurality of fixation means for fixing said sizing plate to the bone end, said extraction means centrally positioned with respect to said fixation means.

11. The orthopaedic instrumentation assembly of claim 10, wherein said fixation means comprises:
a plurality of fixation pins, and wherein said sizing plate includes a plurality of apertures corresponding in number to said fixation pins.

12. The orthopaedic instrumentation assembly of claim 7, further comprising:
a plurality of fixation pins, said sizing plate having a plurality of apertures, said apertures having counterbores on said second surface of said sizing plate.

13. A method for removing an orthopaedic plate member from a bone end comprising the steps of:
inserting a plate extractor key into an opening defined through the plate member;
moving said extractor key into position to engage at least a portion of said extractor key in a recess defined between the plate member and the bone end; and
pulling said extractor key away from the bone end.

14. The method of claim 10, wherein said pulling step comprises:
securing an extraction instrument to said extractor key; and
pulling said extraction instrument away from the bone end.
